(19) United States
(12) Patent Application Publication
(10) Pub. No.: US 2023/0329675 A1
(43) Pub. Date: Oct. 19, 2023

(54) ULTRASONIC DIAGNOSTIC APPARATUS, IMAGE GENERATION PROCESSING METHOD, AND IMAGE GENERATION PROCESSING PROGRAM

(71) Applicant: Konica Minolta, Inc., Tokyo (JP)

(72) Inventor: TOMOHITO SAKAI, Yokohama-shi (JP)

(21) Appl. No.: 18/130,168

(22) Filed: Apr. 3, 2023

(30) Foreign Application Priority Data
Apr. 13, 2022 (JP) 2022-066497

(51) Int. Cl. A61B 8/00; A61B 8/08

(52) U.S. Cl. CPC A61B 8/463; A61B 8/488

(57) ABSTRACT

An ultrasonic diagnostic apparatus includes: a hardware processor that generates an ultrasonic image of a subject based on a first image generation condition; and generates an enlarged image of a region of interest set in the ultrasonic image based on a second image generation condition; and an input part capable of separately setting and inputting the first image generation condition and the second image generation condition.

The entire disclosure of Japanese patent Application No. 2022-066497, filed on Apr. 13, 2022, is incorporated herein by reference in its entirety.

### BACKGROUND

### Technological Field

The present invention relates to an ultrasonic diagnostic apparatus, an image generation processing method, and an image generation processing program.

### Description of the Related Art

Conventionally, as one of medical image diagnostic apparatuses, there has been known an ultrasonic diagnostic apparatus that transmits an ultrasonic wave toward a subject, receives a reflected wave of the ultrasonic wave, and performs predetermined signal processing on a reception signal to visualize the shape, property, or dynamics inside the subject as an ultrasonic image Since the ultrasonic diagnostic apparatus can acquire an ultrasonic image by a simple operation of applying an ultrasonic probe to a body surface or inserting the ultrasonic probe into a body, the ultrasonic diagnostic apparatus is safe, and a burden on a subject is small.

In an ultrasonic diagnostic apparatus of an electronic scanning system, for example, a phased array technology capable of controlling a beam direction of an ultrasonic wave and a shape of the ultrasonic wave by electronically changing drive timing of each transducer using an ultrasonic probe (so-called array probe) in which a plurality of transducers is arranged in an array is used. In the electronic scanning system, a transducer group including a plurality of continuous transducers is sequentially shifted and driven in an array direction of the transducers. Thus, a diagnosis target can be scanned along the array direction of the transducers (hereinafter, referred to as a “scanning direction”).

In addition, as an example of an electronic scanning system, trapezoidal scanning (trapezoid scanning) in which a beam direction of an ultrasonic wave is changed has been put into practical use. By performing such a trapezoidal scanning, it is possible to perform scanning in a range wider than the entire width of the plurality of transducers, and thus, it is possible to enlarge the diagnosis region in the ultrasonic diagnostic apparatus.

Meanwhile, there is a case where a partial region of an ultrasonic image generated by an ultrasonic diagnostic apparatus is set as a region of interest. Considering that the portion of the region of interest is, for example, a portion requiring detailed observation in the ultrasonic image by the user, it is desirable that the portion of the region of interest be an image with high resolution.

For example, JP H6-217981 A attempts to improve the image quality of the region of interest by performing control to increase the number of scanning lines in the region of interest. In addition, in JP 2011-239906 A, when the ultrasonic image is enlarged, the number of sampling points on each scanning line is increased, the density of the scanning line is increased, and the like to suppress blurring when the ultrasonic image is enlarged.

However, the range in which the ultrasonic image for performing the ultrasonic diagnosis is generated and the range in which the region of interest is set vary depending on the subject. In the configurations described in JP H6-217981 A and JP 2011-239906 A, since the generation condition of the ultrasonic image is limited, the usability is poor for the user.

### SUMMARY

An object of the present invention is to provide an ultrasonic diagnostic apparatus, an image generation processing method, and an image generation processing program capable of improving the usability of a user.

To achieve the abovementioned object, according to an aspect of the present invention, an ultrasonic diagnostic apparatus reflecting one aspect of the present invention comprises: a hardware processor that generates an ultrasonic image of a subject based on a first image generation condition; and generates an enlarged image of a region of interest set in the ultrasonic image based on a second image generation condition; and an input part capable of separately setting and inputting the first image generation condition and the second image generation condition.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a view illustrating an appearance of an ultrasonic diagnostic apparatus according to an embodiment of the present invention;

FIG. 2 is a view illustrating an example of a scannable range in an ultrasonic probe;

FIG. 3 is a block diagram illustrating a main part of a control system of the ultrasonic diagnostic apparatus;

FIG. 4 is a view for explaining an acoustic line angle;

FIG. 5 is a view illustrating an example of a scannable range in trapezoid scanning;

FIG. 6 is a view illustrating an example of a scannable range in trapezoid scanning;

FIG. 7 is a flowchart illustrating an example of an operation example of image generation control in a control unit; and

FIG. 8 is a block diagram illustrating a main part of a control system of an ultrasonic diagnostic apparatus according to a modification.

### DETAILED DESCRIPTION OF EMBODIMENTS

Hereinafter, one or more embodiments of the present invention will be described with reference to the drawings. However, the scope of the invention is not limited to the disclosed embodiments.

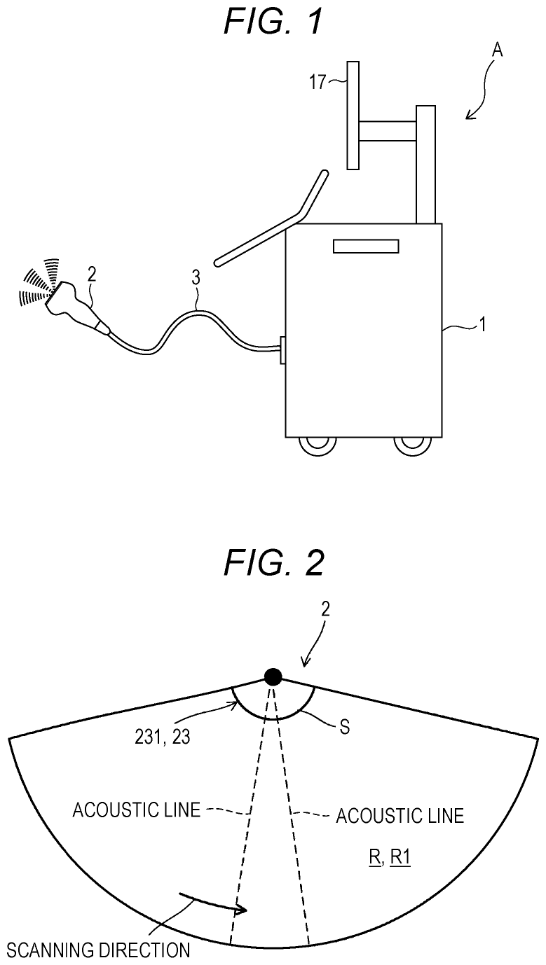

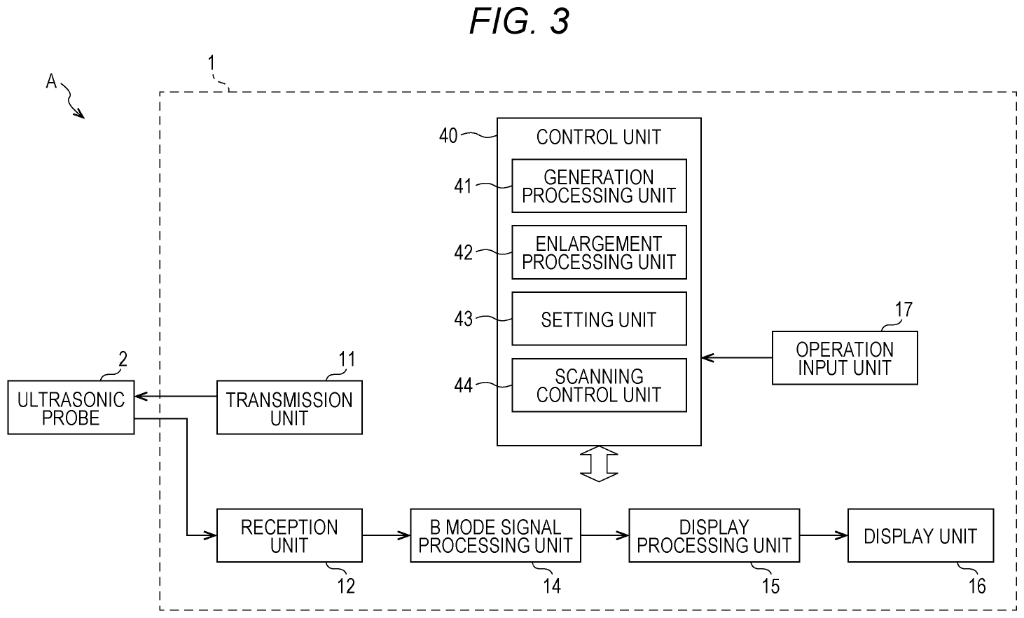

FIG. 1 is a view illustrating an appearance of an ultrasonic diagnostic apparatus A according to an embodiment of the present invention. FIG. 2 is a view illustrating an example of a scannable range in an ultrasonic probe 2. FIG. 3 is a block diagram illustrating a main part of a control system of the ultrasonic diagnostic apparatus A.

As illustrated in FIG. 1, the ultrasonic diagnostic apparatus A includes an ultrasonic diagnostic apparatus main body 1 and the ultrasonic probe 2. The ultrasonic diagnostic apparatus main body 1 and the ultrasonic probe 2 are connected via a cable 3. Note that the ultrasonic probe 2 may be connected to the ultrasonic diagnostic apparatus main body 1 via wireless communication.

The ultrasonic diagnostic apparatus A is used to visualize the shape, property, or dynamics inside the subject as an ultrasonic image and perform image diagnosis. The ultrasonic diagnostic apparatus A has a B mode for displaying only a B mode image as a display mode. Note that the ultrasonic diagnostic apparatus A may have a CFM mode in which a color flow mapping (CFM) image obtained by a color Doppler method is superimposed and displayed on a B mode image.

The ultrasonic probe 2 transmits an ultrasonic wave to the subject, receives an ultrasonic echo reflected by the subject, converts the ultrasonic echo into a reception signal, and transmits the reception signal to the ultrasonic diagnostic apparatus main body 1. The ultrasonic probe 2 is a probe compatible with an electronic scanning system, and for example, a linear probe, a convex probe, or a sector probe can be applied. In the present embodiment, a case will be described in which an ultrasonic probe capable of supporting a wider diagnosis region (for example, a convex probe) is applied as the ultrasonic probe 2.

As illustrated in FIG. 2, the ultrasonic probe 2 includes a transducer array 23. The transducer array 23 includes a plurality of transducers 231 arranged in the scanning direction.

The plurality of transducers 231 is arranged in a manner that transducer surfaces S are arranged on an arc. Therefore, the scanning direction is a direction along the arc including the transducer surface S (for example, a counterclockwise direction in the drawing). With such the transducer array 23, a diagnosis region R in the ultrasonic diagnostic apparatus A becomes a fan-shaped region. Note that, in FIG. 2, each of the plurality of transducers 231 is indicated by a line connecting the plurality of transducers with a curve.

According to the ultrasonic probe 2, the ultrasonic wave can be converged in the scanning direction (so-called electronic focus) by sequentially switching the transducer 231 driven in the scanning direction.

Note that acoustic lines corresponding to the number of transducers 231 pass through the diagnosis region R, but in FIG. 2, only two acoustic lines of ultrasonic waves are illustrated in consideration of the visibility of the drawing. In addition, the two acoustic lines illustrated in FIG. 2 correspond to two transducers 231 adjacent to each other in the scanning direction among the plurality of transducers 231.

The ultrasonic diagnostic apparatus main body 1 visualizes the internal state of the subject as an ultrasonic image using a reception signal from the ultrasonic probe 2. As illustrated in FIG. 3, the ultrasonic diagnostic apparatus main body 1 includes a transmission unit 11, a reception unit 12, a B mode signal processing unit 14, a display processing unit 15, a display unit 16, an operation input unit 17, a control unit 40, and the like.

The transmission unit 11, the reception unit 12, the B mode signal processing unit 14, and the display processing unit 15 include, for example, at least one dedicated hardware (electronic circuit) corresponding to each processing such as a digital signal processor (DSP), an application specific integrated circuit (ASIC), or a programmable logic device (PLD).

The control unit 40 includes a central processing unit (CPU) as an arithmetic/control device, a random access memory (RAM) as a main storage device, a read only memory (ROM), and the like. The ROM stores a basic program and basic setting data. The CPU reads a program corresponding to the processing content from the ROM, develops the program in the RAM, and executes the developed program, centrally controlling the operation of each functional block (the transmission unit 11, the reception unit 12, the B mode signal processing unit 14, the display processing unit 15, and the display unit 16) of the ultrasonic diagnostic apparatus main body 1.

In the present embodiment, the function of each functional block is realized by cooperation between each hardware constituting the functional block and the control unit 40. Note that a part or all of the functions of each functional block may be realized by the control unit 40 executing the program, or each functional block may have a configuration capable of executing the program.

The control unit 40 includes a generation processing unit 41, an enlargement processing unit 42, a setting unit 43, and a scanning control unit 44.

The generation processing unit 41 generates an ultrasonic image of the subject based on the first image generation condition. The enlargement processing unit 42 generates an enlarged image of the region of interest set in the ultrasonic image based on the second image generation condition.

The first image generation condition and the second image generation condition are conditions for generating an image, and include at least one of a transmission frequency, a transmission focal depth, the number of transmission focal points, a reception band setting, a scanning method, a sound speed, a sound linear density, a frequency compound, a spatial compound, image processing, a spatial filter, a temporal filter, a dynamic range, and a gain.

The region of interest is a partial region of the ultrasonic image, and is, for example, a region that is a portion where the observer (user) desires detailed observation in the diagnosis region of the ultrasonic diagnostic apparatus A.

The setting unit 43 sets the region of interest of the ultrasonic image based on an input of the operation input unit 17 to be described later.

The scanning control unit 44 sets the scanning method of the ultrasonic probe 2 based on the image generation condition input by the operation input unit 17.

Examples of the scanning method include a normal scanning method (hereinafter, normal scanning) and a trapezoid scanning method (hereinafter, trapezoid scanning). In the normal scanning and the trapezoid scanning, an acoustic line angle θ to be transmitted and received is included in the scanning condition.

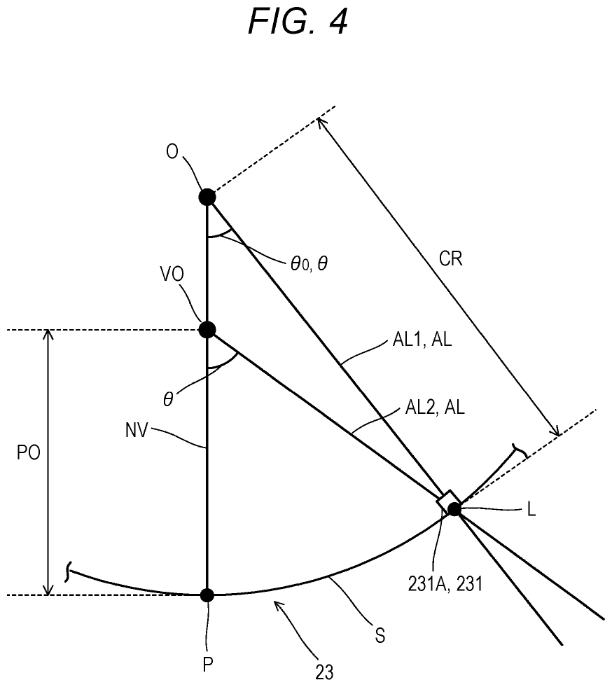

As illustrated in FIG. 4, the acoustic line angle θ is an angle formed by an acoustic line AL and a center normal line NV of the transducer surface S. The acoustic line AL is a center line of each beam of each transducer 231. The center normal line NV is a normal line passing through a probe origin O, which is the center of the arc of the transducer surface S, and a center position P in the scanning direction of the arc formed by the transducer surface S.

The acoustic line angle θ is set for each beam emission point L of each transducer 231, and is expressed by an angle with a symbol (±) based on the center normal line NV. Specifically, the acoustic line angle θ is represented by, for example, a symbol + on the right side with respect to the center normal line NV, and a symbol − on the left side with respect to the center normal line NV. In addition, the beam emission point L is a point at which the acoustic line AL of the ultrasonic wave intersects the transducer surface S.

The normal scanning is a scanning method (first scanning method) based on the probe origin O. In the normal scanning, a tangent line passing through the beam emission point L and an acoustic line AL1 corresponding to the beam emission point L perpendicularly intersect, and the acoustic line AL1 of each transducer 231 intersects at the probe origin O (first predetermined point). In addition, since the plurality of transducers 231 is arranged at equal intervals, inter-acoustic line angles AO of the two adjacent acoustic lines AL are all equal. The inter-acoustic line angle Δθ is an angle formed by the upstream acoustic line AL and the downstream acoustic line AL in the scanning direction.

The trapezoid scanning is, for example, a scanning method based on a virtual origin VO on the center normal line NV. In the trapezoid scanning, for example, a point at which the acoustic line AL2 and the center normal line NV in a predetermined transducer 231A intersect is the virtual origin VO.

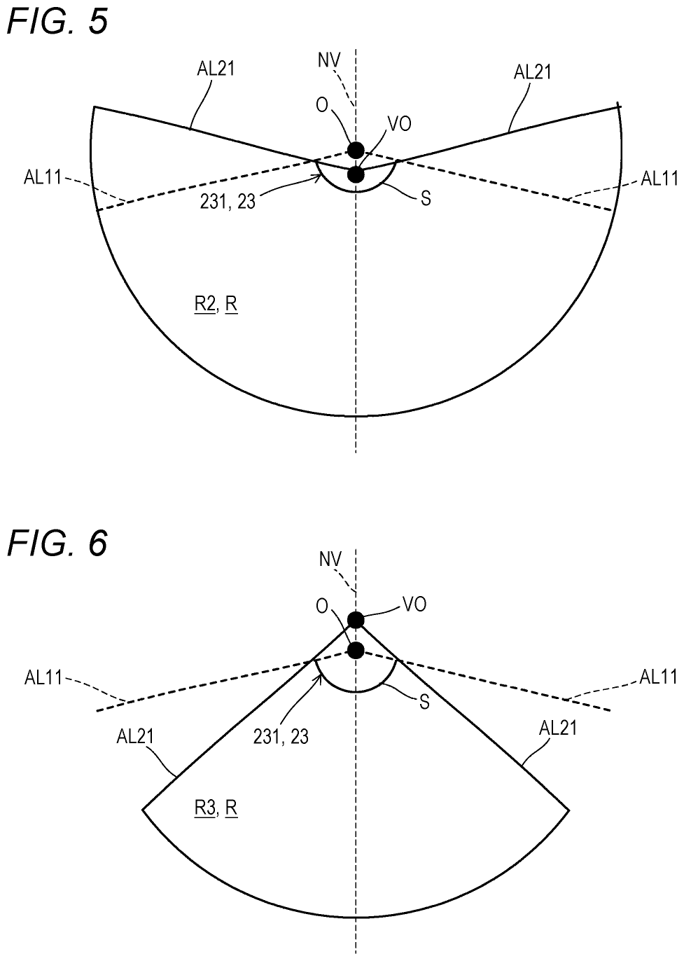

In the trapezoid scanning, since the virtual origin VO shifts from the probe origin O, the inter-acoustic line angle Δθ hardly becomes uniform between each acoustic line. For example, as illustrated in FIG. 5, in a case where the virtual origin VO is shifted toward the transducer 231 side (− side) with respect to the probe origin O, the inter-acoustic line angle Δθ is more likely to spread than in the normal scanning.

In this case, an acoustic line AL21 to the transducer 231 at the end portion in the scanning direction is positioned above an acoustic line AL11 to the transducer 231 at the end portion in the normal scanning. Therefore, in the trapezoid scanning, the fan shape (R2 portion) of the diagnosis region R can be enlarged more than the fan shape (R1 portion in FIG. 2) of the diagnosis region of the normal scanning.

In addition, as illustrated in FIG. 6, in a case where the virtual origin VO is shifted toward the transducer 231 side (+ side) with respect to the probe origin O, the inter-acoustic line angle Δθ is more likely to narrower than in the normal scanning. In this case, the acoustic line AL21 to the transducer 231 at the end portion in the scanning direction is positioned below the acoustic line AL11 to the transducer 231 at the end portion in the normal scanning. Therefore, in the trapezoid scanning, the fan shape (R3 portion) of the diagnosis region R can be made smaller than the fan shape (R1 portion in FIG. 2) of the diagnosis region of the normal scanning.

There are two types of trapezoid scanning methods, namely, a virtual origin point fixed type method and a virtual origin point moving type method, and can be appropriately set according to the setting range of the diagnosis region.

By using the trapezoid scanning as described above, the scannable range of the ultrasonic diagnostic apparatus A can be set to a diagnosis region wider or narrower than a diagnosis region R1 (see FIG. 2) of the normal scanning, and furthermore, ultrasonic images of various ranges can be generated.

As illustrated in FIG. 3, the transmission unit 11 generates a transmission signal (drive signal) in accordance with an instruction from the control unit 40, and outputs the transmission signal to the ultrasonic probe 2. Specifically, the transmission unit 11 controls driving of the ultrasonic probe 2 based on the scanning method set by the control unit 40. Although not illustrated, the transmission unit 11 includes, for example, a clock generation circuit, a pulse generation circuit, a pulse width setting unit, and a delay circuit.

The clock generation circuit generates a clock signal that determines a transmission timing and a transmission frequency of the pulse signal. The pulse generation circuit generates a bi-polar rectangular wave pulse having a preset voltage amplitude at a predetermined period. The pulse width setting unit sets the pulse width of the rectangular wave pulse output from the pulse generation circuit. The rectangular wave pulse generated by the pulse generation circuit is separated into different wiring paths for each transducer 231 of the ultrasonic probe 2 before or after being input to the pulse width setting unit. The delay circuit delays the generated rectangular wave pulse according to the drive timing of each transducer 231 and outputs the delayed rectangular wave pulse to the ultrasonic probe 2.

By controlling the drive timing of the transducer 231, the acoustic line angles θ of a plurality of ultrasonic waves transmitted in one scan can be made different.

In accordance with an instruction from the control unit 40, the reception unit 12 receives a reception signal from the ultrasonic probe 2 and outputs the reception signal to the B mode signal processing unit 14. Although not illustrated, the reception unit 12 includes, for example, an amplifier, an A/D conversion circuit, and a phasing addition circuit.

The amplifier amplifies the reception signal corresponding to the ultrasonic wave received by each transducer 231 of the ultrasonic probe 2 at a predetermined amplification factor set in advance. The A/D conversion circuit converts the amplified reception signal into digital data at a predetermined sampling frequency. The phasing addition circuit applies a delay time to the A/D-converted reception signal for each wiring path corresponding to the transducer 231 to adjust the time phase, and adds them (phasing addition).

In accordance with an instruction from the control unit 40, the B mode signal processing unit 14 performs envelope detection processing, logarithmic compression processing, and the like on the reception data for the B mode image from the reception unit 12, performs adjustment of the dynamic range and the gain, and performs luminance conversion, generating B mode image data.

In accordance with an instruction from the control unit 40, the display processing unit 15 converts the image data generated by the B mode signal processing unit 14 into a display signal corresponding to the display unit 16 and outputs the display signal, and causes the display unit 16 to display the B mode image Note that the display processing unit 15 includes a digital scan converter (DSC) that performs coordinate conversion and pixel interpolation according to the type of the ultrasonic probe 2.

In addition, when receiving a command to enlarge and display the region of interest from the operation input unit 17 and the like, the display processing unit 15 outputs a display signal to enlarge and display image data corresponding to the region of interest to the display unit 16.

The display unit 16 includes, for example, a liquid crystal display, an organic EL display, a CRT display, and the like. The display unit 16 displays an image based on a display signal from the display processing unit 15 in accordance with an instruction from the control unit 40.

The operation input unit 17 is, for example, a user interface that receives an input of information regarding diagnosis. The operation input unit 17 includes, for example, an operation panel having a plurality of input switches, a keyboard, a mouse, and the like. The user can set the region of interest, the diagnosis site, the type of the ultrasonic probe 2, and the like via the operation input unit 17.

Note that an external device (for example, a tablet terminal) communicably connected to the ultrasonic diagnostic apparatus main body 1 can also be applied to at least one of the display unit 16 and the operation input unit 17.

In addition, the operation input unit 17 can separately set and input the first image generation condition corresponding to the ultrasonic image and the second image generation condition corresponding to the enlarged image. That is, in the present embodiment, each of the image generation conditions of the ultrasonic image and the enlarged image can be set by the user's input.

For example, the enlarged image is shallower than the ultrasonic image. Therefore, if the transmission frequency and the reception band setting in the second image generation condition are changed to a higher frequency side than the first image generation condition, the image quality can be improved.

In addition, since the enlarged image has a smaller scanning range than the ultrasonic image, the surface temperature and the acoustic output of the probe become high. Therefore, the transmission energy is limited, and as a result, a signal to noise ratio (SNR) decreases, and thus, a decrease in contrast occurs. Therefore, SNR can be compensated by lowering the dynamic range in the second image generation condition to be lower than that in the first image generation condition.

In addition, since the enlarged image has a smaller scanning range than the ultrasonic image, the frame rate increases. Therefore, in the second image generation condition, noise can be reduced by making the temporal filter stronger than that in the first image generation condition.

In addition, in the enlarged image, the ratio of speckles to the image size changes more than in the ultrasonic image. Therefore, image quality can be improved by changing image processing in order to favorably suppress speckles in the enlarged image.

In the present embodiment, by separately setting the first image generation condition and the second image generation condition, it is possible to generate an ultrasonic image and an enlarged image suitable for purposes such as image quality improvement and noise reduction.

For example, in a case where an ultrasonic image is observed, the user sets and inputs the first image generation condition to the operation input unit 17 according to the range of the diagnosis region to be observed, the image quality of the ultrasonic image, and the acceptable level of noise.

The control unit 40 that has acquired the first image generation condition set and input by the operation input unit 17 sets the image generation condition based on the first image generation condition, for example, the scanning control unit 44 sets the scanning method to be the range of the diagnosis region corresponding to the first image generation condition.

It is assumed that the generation processing unit 41 generates an ultrasonic image by the scanning method set by the control unit 40, the user observes the ultrasonic image displayed on the display unit 16, and sets the region of interest via the operation input unit 17.

The user sets and inputs a second image generation condition of the enlarged image related to the region of interest via the operation input unit 17. For example, the user sets and inputs an image generation condition different from the first image generation condition as the second image generation condition from the viewpoint of image quality improvement, noise reduction, and the like.

The second image generation condition may be, for example, a condition in which image processing and the like corresponding to an image (enlarged image) smaller than the ultrasonic image is performed, such as the above-described image quality condition and noise reduction condition. Such a condition can be set by adjusting at least one of the transmission frequency, the transmission focal depth, the number of transmission focal points, the reception band setting, the scanning method, the sound speed, the acoustic line density, the frequency compound, the spatial compound, the image processing, the spatial filter, the temporal filter, the dynamic range, and the gain between the first image generation condition and the second image generation condition.

The control unit 40 that has acquired the second image generation condition set and input by the operation input unit 17 sets, for example, an image generation condition based on the second image generation condition.

The enlargement processing unit 42 generates an enlarged image under the second image generation condition set by the control unit 40.

In this way, in the present embodiment, it is possible to generate what the user desires in both the ultrasonic image and the enlarged image. As a result, the usability of a user can be improved.

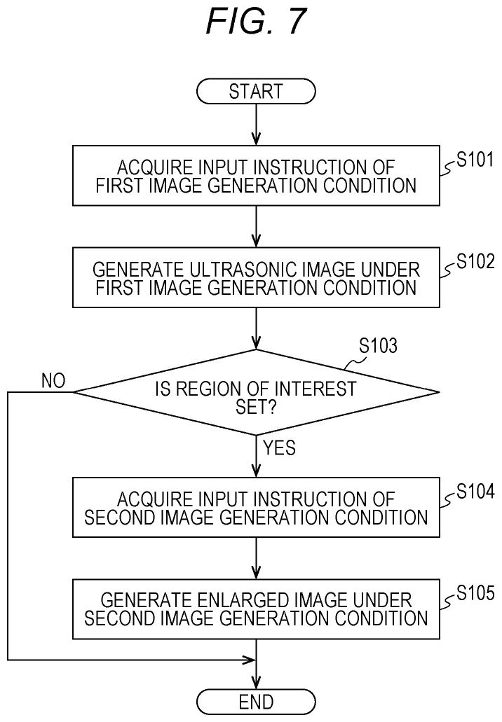

Next, an operation example when image generation control is executed in the control unit 40 will be described. FIG. 7 is a flowchart illustrating an example of an operation example of image generation control in the control unit 40. The processing in FIG. 7 is appropriately executed, for example, when an image is generated by the ultrasonic diagnostic apparatus A for the subject.

As illustrated in FIG. 7, the control unit 40 acquires an input instruction of the first image generation condition (step S101). After that, the control unit 40 generates an ultrasonic image under the first image generation condition (step S102).

Next, the control unit 40 determines whether or not a region of interest is set in the ultrasonic image generated in step S102 (step S103). As a result of the determination, in a case where there is no setting of the region of interest (step S103, NO), this control ends.

On the other hand, in a case where the region of interest is set (step S103, YES), the control unit 40 acquires an input instruction of the second image generation condition (step S104). After that, the control unit 40 generates an enlarged image under the second image generation condition (step S105). After step S105, this control ends.

According to the present embodiment configured as described above, since both the first image generation condition for the ultrasonic image and the second image generation condition for the enlarged image can be set by the user's input, both the ultrasonic image and the enlarged image can be generated under conditions reflecting the user's intention. As a result, an ultrasonic image and an enlarged image desired by a user can be easily obtained, in a manner that the usability of a user can be improved.

In addition, since the image generation condition can be input through the user interface, the image generation condition can be set by a simple operation.

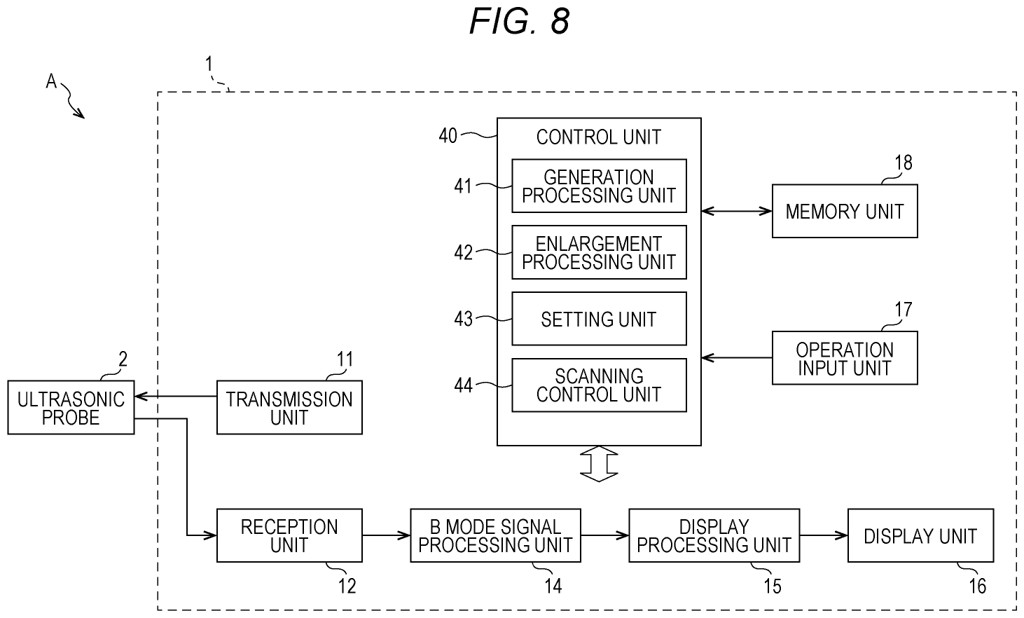

Note that, in the above embodiment, the first image generation condition and the second image generation condition set by the user may be stored. That is, as illustrated in FIG. 8, for example, the control unit 40 may independently store the first image generation condition and the second image generation condition set by the user in advance in a memory unit 18 (storage) of the ultrasonic diagnostic apparatus A.

In this way, for example, in a case where the same image of the subject is re-generated, a case where the same image generation condition is applied to a plurality of subjects, and the like, it is possible to save the user from re-inputting the same image generation condition.

In addition, in the above embodiment, the first image generation condition and the second image generation condition correspond to the B mode. However, the present invention is not limited to this, and the image generation condition may correspond to at least one image mode of the B mode, the M mode, the color Doppler mode, the power Doppler mode, the CW mode, and the elastography mode.

In addition, in the above embodiment, the first image generation condition and the second image generation condition can be input and set on the same user interface (operation input unit 17), but the present invention is not limited to this. For example, the first image generation condition and the second image generation condition may be input-settable with different user interfaces.

In addition, in the above embodiment, the first image generation condition and the second image generation condition are different conditions, but the present invention is not limited to this, and the same conditions may be used as long as the conditions are input and set by the user.

In addition, in the above embodiment, both the normal scanning method and the trapezoid scanning method can be applied, but the present invention is not limited to this, and only one of the normal scanning method and the trapezoid scanning method may be applied.

In addition, in the above embodiment, the ultrasonic probe having the arc-shaped transducer surface has been exemplified, but the present invention is not limited to this, and the ultrasonic probe may have a transducer surface other than the arc-shaped transducer surface, such as a linear transducer surface.

Although embodiments of the present invention have been described and illustrated in detail, the disclosed embodiments are made for purposes of illustration and example only and not limitation. The scope of the present invention should be interpreted by terms of the appended claims That is, the present invention can be implemented in various forms without departing from the gist or main features.

What is claimed is:

1. An ultrasonic diagnostic apparatus comprising:
a hardware processor that generates an ultrasonic image of a subject based on a first image generation condition; and generates an enlarged image of a region of interest set in the ultrasonic image based on a second image generation condition; and
an input part capable of separately setting and inputting the first image generation condition and the second image generation condition.

2. The ultrasonic diagnostic apparatus according to claim 1, wherein
the input part is a user interface.

3. The ultrasonic diagnostic apparatus according to claim 1, comprising:
a storage capable of independently storing the first image generation condition and the second image generation condition in advance.

4. The ultrasonic diagnostic apparatus according to claim 1, wherein
the first image generation condition and the second image generation condition include at least one of a transmission frequency, a transmission focal depth, a number of transmission focal points, a reception band setting, a scanning method, a sound speed, an acoustic line density, a frequency compound, a spatial compound, image processing, a spatial filter, a temporal filter, a dynamic range, and a gain.

5. The ultrasonic diagnostic apparatus according to claim 1, wherein
the first image generation condition and the second image generation condition are image generation conditions corresponding to at least one image mode of a B mode, an M mode, a color Doppler mode, a power Doppler mode, a CW mode, and an elastography mode.

6. An image generation processing method of an ultrasonic diagnostic apparatus including an input part capable of separately setting and inputting a first image generation condition and a second image generation condition, the image generation processing method comprising:
generating an ultrasonic image of a subject based on the first image generation condition input from the input part; and
generating an enlarged image of a region of interest set in the ultrasonic image based on the second image generation condition input from the input part.

7. A non-transitory recording medium storing a computer readable image generation processing program of an ultrasonic diagnostic apparatus including an input part capable of separately setting and inputting a first image generation condition and a second image generation condition, the image generation processing program causing a computer to execute:
generating an ultrasonic image of a subject based on the first image generation condition input from the input part; and
generating an enlarged image of a region of interest set in the ultrasonic image based on the second image generation condition input from the input part.
